(19) United States
(12) Patent Application Publication
(10) Pub. No.: US 2010/0205000 A1
(43) Pub. Date: Aug. 12, 2010

(54) Management System For Public Facilities

(76) Inventor: Young Bin Cho, Gyeonggi-do (KR)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/677,072

(22) PCT Filed: Oct. 17, 2008
(86) PCT No.: PCT/KR08/06161

§ 371 Date: Mar. 8, 2010

(30) Foreign Application Priority Data
Oct. 18, 2007 (KR) 10-2007-0104939
Nov. 2, 2007 (KR) 10-2007-0111420
Nov. 27, 2007 (KR) 10-2007-0121196

(51) Int. Cl. G06Q 10/00; B60Q 1/48; G08B 5/22; G06Q 50/00; G06Q 30/00; H04Q 5/22

(52) U.S. Cl. 705/2; 340/932.2; 340/825.49; 705/13; 705/14.38; 705/28; 235/375

(57) ABSTRACT

Disclosed is a management system for public facilities. The management system of public facilities comprises a tag unit which transmits identification information of a target resource to be managed through a first network, an intermediate managing unit which has intrinsic position information thereof, processes the identification information received through the first network, and transmits the identification information through a second network, and a host unit which creates state information comprising a movement state of the target resource based on the intrinsic position information of the intermediate managing unit and the identification information of the target resource received through the second network. The public facilities used by many persons can be easily monitored and effectively managed through short range wireless technologies.

### TECHNICAL FIELD

The present invention relates to a management system for public facilities.

### BACKGROUND ART

A ubiquitous network technology allows a user to freely access various networks regardless of time and location. Such a ubiquitous network technology can be realized through various schemes using communication technologies, such as telematics communication, short range wireless communication, and mobile communication technologies.

Recently, short range wireless technologies such as ZigBee and a radio frequency identification (RFID) have been developed based on the IEEE 802.15.4 standard. However, the above technologies are in the early stage of development, so application fields thereof are limited.

### DISCLOSURE OF INVENTION

### Technical Problem

An object of the present invention is to provide a management system of public facilities, capable of easily monitoring and effectively managing situations of facilities (e.g., illuminators), articles, and users in the public facilities such as a parking lot, a hospital, and a shopping center used by many persons through short range wireless technologies.

### Technical Solution

According to the embodiment, a management system of public facilities, comprises a tag unit which transmits identification information of a target resource to be managed through a first network, an intermediate managing unit which has intrinsic position information thereof, processes the identification information received through the first network, and transmits the identification information through a second network, and a host unit which creates state information comprising a movement state of the target resource based on the intrinsic position information of the intermediate managing unit and the identification information of the target resource received through the second network.

### ADVANTAGEOUS EFFECTS

According to the present invention, public facilities used by many persons can be easily monitored and effectively managed through short range wireless technologies.

### BRIEF DESCRIPTION OF DRAWINGS

FIG. 1 is a control block diagram schematically showing a management system for public facilities according to an embodiment of the present invention;

FIGS. 2A to 2C are schematic views showing a network topology according to an embodiment of the present invention;

FIG. 3 is a view showing the structure of a protocol stack of data transmitted/received through a second network in a management system of public facilities according to an embodiment of the present invention;

FIG. 4 is a data structure view showing the form of a data packet processed in a ZigBee device of in management system for public facilities according to an embodiment of the present invention;

FIG. 5 is a control block diagram showing an RFID tag device in a management system for public facilities according to an embodiment of the present invention;

FIG. 6 is a control block diagram showing an RFID reader device in a management system for public facilities according to an embodiment of the present invention;

FIG. 7 is a control block diagram showing a ZigBee device in a management system for public facilities according to an embodiment of the present invention;

FIG. 8 is a control block diagram showing a management system for public facilities according to a first embodiment of the present invention;

FIG. 9 is a control block diagram showing a host unit in the management system of public facilities according to a first embodiment of the present invention;

FIG. 10 is a control block diagram showing a parking information guiding device in a management system of public facilities according to a first embodiment of the present invention;

FIG. 11 is a control block diagram showing a tag unit in a management system of public facilities according to a first embodiment of the present invention;

FIG. 12 is a view showing an example of a guide image provided by a tag unit in a management system of public facilities according to a first embodiment of the present invention;

FIG. 13 is a control block diagram showing a management system of public facilities according to a second embodiment of the present invention;

FIG. 14 is a control block diagram showing a host unit in a management system of public facilities according to a second embodiment of the present invention;

FIGS. 15A to 15D are views showing images displayed by an information providing terminal in a management system for public facilities according to a second embodiment of the present invention;

FIG. 16 is a control block diagram showing a management system of public facilities according to a third embodiment of the present invention;

FIG. 17 is a control block diagram showing a third ZigBee device in a management system of public facilities according to a third embodiment of the present invention;

FIG. 18 is a circuit diagram showing a power supply of a shelf connected to a shelf management device and a shelf display device in a management system of public facilities according to a third embodiment of the present invention;

FIG. 19 is a view showing installation of a shelf management device and a shelf display device according to a third embodiment of the present invention; and

FIG. 20 is a view showing the assembly of a shelf display device and a shelf in a management system of public facilities according to a third embodiment of a present invention.

### BEST MODE FOR CARRYING OUT THE INVENTION

Hereinafter, a communication system according to the embodiment will be described in detail with reference to accompanying drawings.

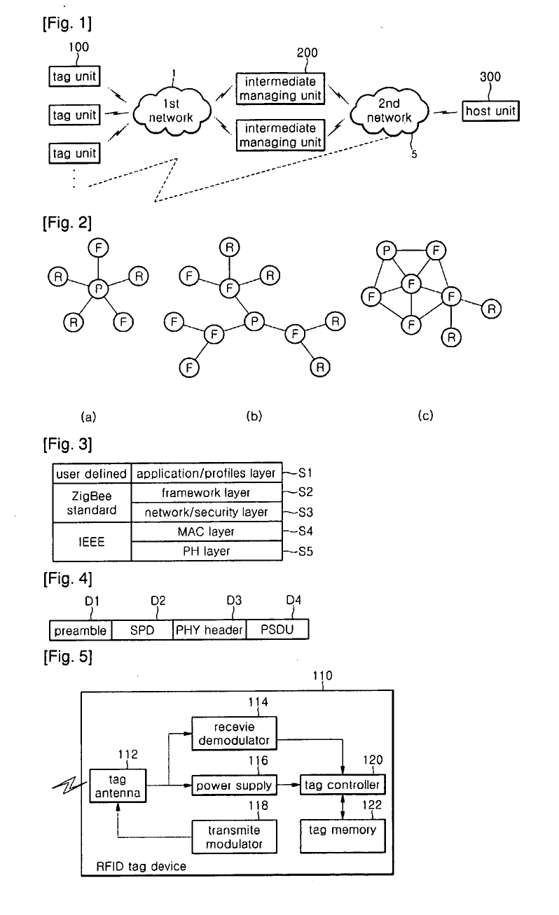

FIG. 1 is a control block diagram schematically showing a management system for public facilities according to an embodiment of the present invention.

As shown in FIG. 1, the management system for public facilities according to an embodiment of the present invention comprises a tag unit 100, an intermediate managing unit 200, and a host unit 300. The tag unit 100 transmits information about identification of a target resource through a first network 1. The intermediate managing unit 200 has information about an intrinsic position thereof, processes the identification information received through the first network 1, and transmits the identification information through a second network 5. The host unit 300 generates state information comprising to the movement state of the target resource based on the identification information of the target resource received through the second network 5 and the position information of the intermediate managing unit 200.

The identification information of the tag unit 100 may be given to moving resources such as an article, a purchaser, a vehicle to be parked, a patient in a hospital, and a member of a staff. The tag unit 100 may transmit the identification information to one of different networks (such as the first and second networks 1 and 5). In this case, the first network 1 may be an RFID network, and the second network 5 is a ZigBee network.

The intermediate managing unit 200 is installed in a fixed position to receive and process identification information transmitted from a plurality of tag units 100. The intermediate managing unit 200 may transmit the received identification information to the host unit 300. Accordingly, the host unit 300 can detect the position of a moving resource based on the installation position of the intermediate managing unit 200.

The intermediate managing unit 200 may control facilities such as illumination equipment and displays according to the identification information transmitted from the tag unit 100, and may control the facilities according to the control of the host unit 300.

The host unit 300 may collect and analyze tag information provided by the intermediate managing unit 200 or directly provided by the tag unit 100 to manage the movement and the state of various resources identified by the tag unit 100.

FIGS. 2A to 2C are a view schematically showing a network topology capable of forming the management system for public facilities according to an embodiment of the present invention, in particular, a network topology based on a ZigBee network.

The tag unit 100, the intermediate managing unit 200, and the host unit 300 comprise components used for ZigBee communication, and may construct a network as shown in FIG. 2.

FIGS. 2A to 2C show a star network, a mesh network, and a cluster tree network, respectively.

In each network, ZigBee devices marked as F, R, and P denote an FFD, an RFD, and a PAN, respectively.

The FFD performs network initialization, node management, and node information storage. If the FFD allows remaining ZigBee devices to construct one of the above three networks, the FFD is called by a PAN coordinator. The FFD can serve as a router and a coordinator, construct the above three-type networks, and communicate with other FFDs or RFDs.

In contrast, the RFD cannot serve as a coordinator, is a target subject to coordinating of the FFD, and can construct only the star network.

Accordingly, a network of the management system for public facilities can be constructed by allowing one of the intermediate managing unit 200 and the host unit 300 to serve as the FFD. The managing unit 200 and the host unit 300 may serve as the RFD, the FFD, or the PAN coordinator according to the type of a program mounted on a processor chip, that is, the type of a program processing data of a network layer (see S3 of FIG. 3) and a framework layer (see S3 of FIG. 3).

FIG. 3 is a view showing the structure of a protocol stack of data transmitted/received through a ZigBee network that is the second network 5 in the management system for public facilities according to one embodiment of the present invention.

As shown in FIG. 3, the protocol stack processed in the ZigBee network comprises a PH (Physical) layer S5, a MAC (Media Access Controller) layer S4, a network/security layer S3, an application framework layer (hereinafter, referred to as framework layer) S2, an application/profiles layer S1. In this case, the PH layer S5 and the MAC layer S4 are based on the IEEE standard, and the network/security layer S3 and the framework layer S2 are based on the ZigBee Alliance standard. The application/profiles layer S1 corresponds to a user defined field. Accordingly, the network of the management system for public facilities may be set and controlled according to the setting of the application/profiles layer S1.

FIG. 4 is a data structure view showing the form of a data packet processed in the ZigBee device of the management system for public facilities according to an embodiment of the present invention.

As shown in FIG. 4, the data packet for ZigBee communication comprises a preamble D1, an SPD (Start of Packet Delimiter) D2, a PHY header D3, and a PSDU (PHY Service Data Unit) D4. The preamble D1 refers to a series of pulse signals that can be analyzed between ZigBee devices in order to synchronize transmission timing.

The SPD D2 notifies the other party of the ZigBee devices that real packet data are commenced and marks an analysis point of the PHY header D3. The PHY header D3 contains analysis information of the PHY layer (physical layer) S5.

The PSDU D4 is a field containing routing information and information related to target resources to be managed in the public facilities. Accordingly, the PSDU D4 contains tag analysis information and tag identification information of the target resources and information related to facilities (e.g., illumination state information) in addition to the routing information.

FIG. 5 is a control block diagram showing an RFID tag device 110 of the management system for the public facilities according to an embodiment of the present invention.

The RFID tag device 110 can transmit identification information about a target resource to be managed, which is contained in the tag unit 100, to an RFID network that is the first network 1. In addition, the RFID tag device 110 can transmit the identification information to a tag-ZigBee device (see 1150 of FIG. 8) transmitting/receiving data through a ZigBee network that is the second network 5.

Referring to FIG. 5, the RFID tag device 110 comprises a tag antenna 112, a receive demodulator 114, a power supply 116, a transmit modulator 118, a tag controller 120, and a tag memory 122.

The tag antenna 112 receives an information request signal through a radio channel or transmits tag identification information which has been subject to signal processing in the transmit modulator 118. The tag antenna 112 comprises a dipole antenna.

The tag memory 122 stores an information code system. The tag controller 120 creates the tag identification information based on the information code system. The information code system comprises header information, reader identification information, error correction information, and the tag identification information.

The receive demodulator 114 demodulates the information request signal into digital data and transmits the digital data to the tag controller 120. The tag controller 120 analyzes codes of the information request signal that has been demodulated to create the tag identification information. In addition, the tag controller 120 comprises a communication protocol to control radio communication with another RFID device.

The transmit modulator 118 modulates a digital signal created in the tag controller 120 into an RF signal and transmits the RF signal through the tag antenna 112.

The power supply 116 supplies driving power of the RFID tag device 110. The power supply 116 may comprises a rechargeable battery, a voltage stabilizing circuit, etc. The power supply 116 may use driving power of the tag unit 100.

The RFID tag device 110 according to the present embodiment can employ a passive tag or an active tag based on an UHF signal having a frequency band of about 400 MHz to about 900 MHz.

Such an RFID tag device 110 transmits the tag identification information, so that the intermediate managing unit 200 or the host unit 300, which has received the tag identification information, can identify a target resource, and tract the position of the target resource.

FIG. 6 is a control block diagram showing an RFID reader 210 of the intermediate managing unit 200 of the management system for the public facilities according to one embodiment of the present invention. The RFID reader 210 can receive and process tag identification information transmitted by the RFID tag device 110.

The RFID reader 210 comprises a reader antenna 212, a demodulator 214, a transceiver 216, a modulator 218, a reader controller 220, and a reader memory 222.

The reader memory 222 stores a code analysis system similarly to the tag memory 122, so that the reader controller 220 uses the code analysis system when analyzing codes. In addition, the reader controller 220 can store tag analysis information in the reader memory 222.

The reader antenna 212 transmits an information request signal or receives tag identification information from an outside through a radio channel.

The transceiver 216 processes tag information having the form of an RF signal to convert the tag information into a base-band signal. When an information request signal created in the reader controller 220 is modulated into an intermediate-frequency signal through the modulator 218, the transceiver 216 converts the information request signal into an RF signal to transmit the RF signal to the reader antenna 212.

The demodulator 214 demodulates tag identification information, which has been subject to signal processing, into digital data to transmit the digital data to the reader controller 220.

The reader controller 220 comprises a communication protocol to control RFID communication, and periodically transmits an information request signal in order to detect the position of the RFID tag device 110.

The reader controller 220 analyzes the tag identification information converted into the digital data in the demodulator 214. At this time, the reader controller 220 changes a data format of the digital data, and performs digital filtering in order to extract desired information from the digital data. Accordingly, the reader controller 220 can identify a target resource to be managed.

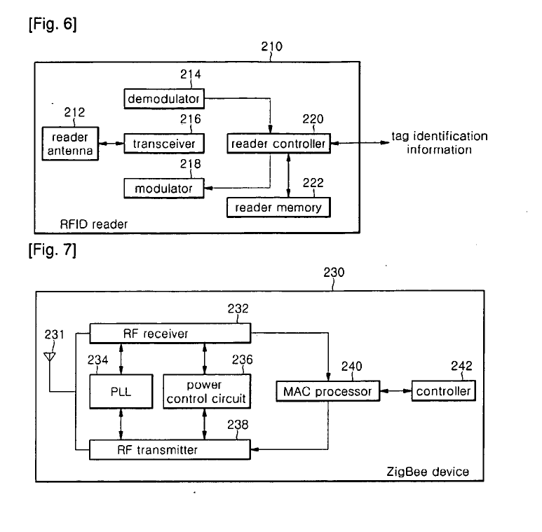

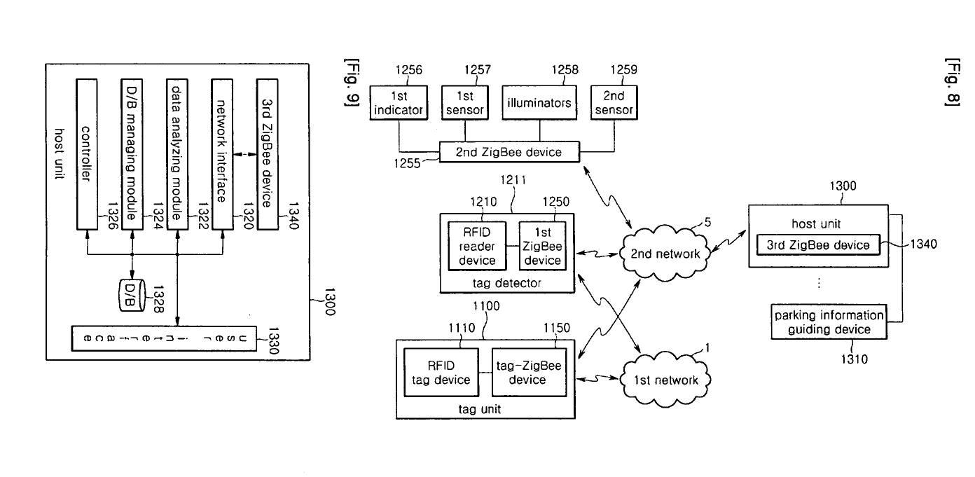

The reader controller 220 may transmit identification information of the target resource to a ZigBee device (see 230 of FIG. 7) belonging to the intermediate managing unit 200.

FIG. 7 is a control block diagram showing the ZigBee device 230 belonging to the intermediate managing unit 200 of the management system for the public facilities according to an embodiment of the present invention.

The ZigBee device 230 comprises an antenna 231, an RF receiver 232, an RF transmitter 238, a phase locked loop (PLL) 234, a power control circuit 236, a MAC processor 240, and a controller 242.

The RF receiver 232, the PLL 234, the RF transmitter 238, and the power control circuit 236 are elements to perform an operation corresponding to the PH layer S5 (see FIG. 3) of the ZigBee protocol stack, and determine an RF communication structure and a network topology.

The RF receiver 232 and the RF transmitter 238 process tag analysis information and tag identification information, which are transmitted from the tag unit 100 and the host unit 300 through a ZigBee communication scheme, into an RF signal and a base-band signal.

The PLL 234 generates an oscillation frequency signal such that the RF receiver 232 and the RF transmitter 238 obtain an intermediate-frequency signal through signal synthesis. The power control circuit 236 adjusts an amount of transmit power by determining the intensity of a received signal.

The RF receiver 232 and the RF transmitter 238 employ a direct sequence spread spectrum (DSSS). In addition, the RF receiver 232 and the RF transmitter 238 employ offset-quadrature phase-shift keying (O-QPSK) with a 32 PN code-length in the case of a 2.4 GHz band, and binary phase-shift keying (BPSK) with a 15 PN code-length in the case of less than a 1 GHz band.

After digital processing is performed in the PH layer S5 (see FIG. 3), the MAC processor 240 analyzes the structure of a data frame, which has been transferred, to approve the data frame and perform error detection with respect to the data frame by using CRC or checksum so that data retransmission is determined. In addition, the MAC processor 240 performs packet routing. The MAC processor 240 is an element to perform an initial hardware network association. The MAC processor 240 provides an optional super-frame structure related to beacons for time synchronization and a guaranteed time slot (GTS) scheme capable of preventing data collision/delay. The MAC processor 240 connects channels with each other through a carrier sense multiple access with collision avoidance (CSMA-CA) scheme.

Meanwhile, the controller 242 constructs a network topology by performing remaining MAC layer functions (which are referred to as Software-MAC corresponding to the above Hardware-MAC), functions of the network/security layer S3 (see FIG. 3), and functions of the framework layer S2 (see FIG. 3), and processes digital data by performing functions of the application/profiles layer S1 (see FIG. 3).

The controller 242 can be connected with the host unit 300 over a cable network comprising a universal asynchronous receiver/transmitter (UART), the Internet (employing TCP/IP), a switch hub, a serial/parallel cable, and can transmit digital data such as tag analysis information, tag identification information, and facility state information to the host unit 300.

FIG. 8 is a control block diagram showing a management system for public facilities according to a first embodiment of the present invention, and, in particular, shows a case in which the present invention is applied to a parking management system.

As shown in FIG. 8, the parking management system according to the first embodiment comprises a tag unit 1100 and a host unit 1300. The tag unit 1100 is attached to a vehicle, and the host unit 1300 creates information about a movement state of the vehicle, a parking state, and an illumination state in parking facilities. In this case, the intermediate managing unit 200 comprises a tag detector 1211, second ZigBee devices 1255, a first sensor 1257, illuminators 1258, a second sensor 1259, and a first indicator 1256 to relay and process data for the purpose of data communication between the tag unit 1100 and the host unit 1300.

The space of the parking facilities may be mainly partitioned into a parking region, a driving region, a parking block, and a parking floor. The parking region refers to a unit region on which a vehicle is parked, and the driving region refers to an region between parking regions, in which the vehicle is moving. In addition, the parking block refers to a group of several parking regions, and the parking floor refers to a floor on which the vehicle is parked.

The illuminators 1258 are uniformly installed on a ceiling of a building to illuminate a predetermined region. One illuminator 1258 is installed in one parking block with the second ZigBee devices 1255. The illuminator 1258 may comprise various devices such as a fluorescent lamp and a light emitting diode (LED), and employs a lower-power LED. Since the illuminators 1258 and the second ZigBee devices 1255 are uniformly installed on the ceiling of the building, the host unit 1300 makes coordinates for the installation regions of the second ZigBee devices 1255.

The first sensor 1257 is a light sensor and installed on a ceiling. The second sensor 1259 can detect a vehicle existing in the parking region, and comprises an ultrasonic sensor, a terrestrial magnetic sensor, a thermal sensor, and a motion sensor. The second sensor 1259 can be mounted on a floor of the parking region.

In the above structure, the first sensor 1257 detects quantity of light to transmit the quantity of the light to the second ZigBee device 1255. The second ZigBee device 1255 controls a driving circuit or power of the illuminator 1258 to turn on/off or dim the illuminator 1258. In addition, if the second sensor 1259 detects a vehicle, the second ZigBee device 1255 can operate the illuminator 1258.

The second sensor 1259 detects whether a vehicle exists in the parking region thereof. The second ZigBee devices 1255 generates information about a parking state of vehicles in the parking region to transmit the information to the host unit 1300, so that the host unit 1300 can monitor all parking regions.

The first indicator 1256 comprises an electric bulletin and an alarm lamp to provide parking information to a person who tries to park. According to the present embodiment, a small-sized first indicator 1256 is installed, and one first indicator 1256 is installed in one parking block.

The tag unit 1100 may be supplied in a parking management office positioned in a gate of the parking facilities when a vehicle enters the parking facilities. In addition, the tag unit 1100 may be realized as a portable device comprising an RFID tag device 1110 and a tag-ZigBee device 1150.

The tag unit 1100 comprises a small-sized display screen to display parking information received in the tag-ZigBee device 1150 to a user. Accordingly, a person can park the vehicle based on the parking state information of vehicles in the parking facilities through the tag unit 1100, and can pay charges for parking by checking parking charge information through the tag unit 1100. The tag unit 1100 comprises a motion sensor to operate in a sleep mode or an activation mode according to the detection result of the motion sensor, so that power consumption is reduced.

The tag detector 1211 communicates with the RFID tag device 1110 of the tag unit 1110 to collect information related to vehicles. The tag detector 1211 comprises an RFID reader device 1210 and a first ZigBee device 1250. The RFID reader device 1210 may comprise a device corresponding to an RFID reader receiving and processing information about an RFID tag. Such the tag detector 1211 may be installed in a gate placed outside a parking lot, a gate placed between a shopping center and a parking lot, or a gate placed between parking floors.

The host unit 1300 comprises a third ZigBee device 1340 performing data communication through the second network 5. Accordingly, the host unit 1300 can transmit/receive data with the tag-ZigBee device 1150 of the tag unit 1110, the first ZigBee device 1250 of the tag detector 1211, and the second ZigBee device 1255 connected to the illuminator 1258 through the third ZigBee device 1340.

The host unit 1300 collects and manages parking information received in the third ZigBee device 1340 to make a database using them. Accordingly, the host unit 1300 transmits the parking information through the third ZigBee device 1340 to the tag-ZigBee device 1150 of the tag unit 1110 so that the parking driver can check the parking information through the tag unit 1110.

The host unit 1300 can provide data to a parking information guiding device 1310 in order to display the parking information to the parking driver or a parking manager.

For example, if the management system for public facilities is installed in a parking lot of a shopping center, a person searches for his/her parked vehicle in the parking lot after shopping. In this case, the parking information guiding device 1310 is installed in a gate placed between the shopping center and the parking to guide information about a position of the parked vehicle, charge information for parking, etc. The parking information guiding device 1310 may be realized in the form of a KIOSK.

In the above structure, when a vehicle that has received the tag unit 1100 is driven in the driving region or enters the parking block to pass through a coverage area of the second ZigBee devices 1255, the second ZigBee devices 1255 can operate the illuminator 1258 while commencing communication with the tag-ZigBee device 1150. The second ZigBee devices 1255 can transmit an on/off state of the illuminator 1258 to the third ZigBee device 1340 of the host unit 1300, so that the host unit 1300 can detect and manage an illumination state of parking facilities.

In addition, when the vehicle that has received the tag unit 1100 moves around the tag detector 1211, the RFID reader device 1210 of the tag detector 1211 receives and analyzes tag identification information from the RFID tag device 1110 of the tag unit 1100. The tag identification information comprises personal information of a driver of who has received the tag unit 1100 and information about the vehicle. For example, the personal information comprises a name, an address, and a phone number of the driver, and the information about the vehicle comprises a type of the vehicle and a registration number of the vehicle. The RFID reader device 1210 of the tag detector 1211 transmits the tag identification information comprising the personal information and the information about the vehicle to the third ZigBee device 1340 of the host unit 1300, and the host unit 1300 makes a database using the received information.

The tag-ZigBee device 1150 of the tag unit 1100 receives tag identification information when passing through the coverage area of the second ZigBee devices 1255, and the second ZigBee devices 1255 transmits the tag identification information to the third ZigBee device 1340.

As described above, the tag-ZigBee device 1150 of the tag unit 1100, the second ZigBee devices 1255, and the third ZigBee device 1340 of the host unit 1300 establish a short range network based on the second network 5. In this case, the tag-ZigBee device 1150 of the tag unit 1100 may comprise an RFD module, and the second ZigBee devices 1255 and the first ZigBee device 1250 of the tag detector 1211 may comprise an FFD module or a PAN coordinator. The third ZigBee device 1340 of the host unit 1300 may comprise a PAN coordinator.

The third ZigBee device 1340 can set a PAN ID and frequency channels of the second ZigBee device 1255 and the first ZigBee device 1250. For example, the parking floor may become a criterion for distinguishing frequency channels, and indicators marked on parking blocks may become a criterion for distinguishing PAN IDs.

When the vehicle equipped with the tag unit 1100 is stopped in a parking region, an indicator given in the parking region may serve as a criterion for distinguishing network (NWK) IDs.

For example, a first channel may be set to a first floor, and a PAN ID of 01 (which are expressed in two bytes) may be set to an A block of the first floor. In addition, an NWK ID of 03 may be set to a third parking region of the A block on the first floor. Accordingly, the ZigBee devices can make ZigBee communication without interference. When a vehicle is parked, the NWK ID can be assigned to the vehicle. Accordingly, information about the vehicle can be recognized. In this case, the vehicle, in detail, a registration number of the vehicle can be identified in cooperation with RFID communication.

In addition, the second ZigBee device 1255 covers a plurality of parking regions in a coverage area thereof. The second ZigBee device 1255 detects tags being communicated with the second ZigBee device 1255 at a present time point and the number of tags entering a sleep mode after making communication with the second ZigBee device 1255 for a predetermined time. Accordingly, the second ZigBee device 1255 can create parking state information comprising information about a full state of parked vehicles in parking blocks and information about the number of empty parking regions. The second ZigBee device 1255 delivers the parking state information to the host unit 1300, and the host unit 1300 can monitor all parking regions.

Accordingly, the host unit 1300 can identify a vehicle passing through a coverage area of the second ZigBee device 1255 by matching personal information and vehicle information, which have already been stored through the tag detector 1211 with tag identification information. The host unit 1300 can store movement information of a vehicle by marking coordinates of the second ZigBee device 1255 delivering information about an identified vehicle on a map.

If the second ZigBee device 1255 determines that the parking region exists in its own parking block, the second ZigBee device 1255 operates the first indicator 1256 to provide a service of guiding information about parking to a driver searching for a parking space. The first indicator 1256 is turned on/off or displays a simple sign such as an arrow, so that the driver can recognize an empty parking region in a remote plate.

The parking information guiding device 1310 receives parking state information, vehicle information, parking time information, and parking charge information and guides the information to a driver returning to his/her parked vehicle after shopping, so that the driver can easily search for his/her parked vehicle and settle and pay charges for parking.

FIG. 9 is a control block diagram showing the host unit 1300 of the management system of the public facilities according to the first embodiment of the present invention, and, particularly, shows a case in which the host unit 1300 is applied to the management system of the public facilities.

As shown in FIG. 9, the host unit 1300 comprises a network interface 1320, a data analyzing module 1322, a database (D/B) managing module 1324, a controller 1326, a D/B 1328, and a user interface 1330. The network interface 1320 connects the third ZigBee device 1340 with the parking information guiding device 1310.

The network interface 1320 receives illumination state information, parking state information, tag analysis information (personal information, vehicle information, etc.), and tag identification information from the second ZigBee device 1255 to form digital data using the information.

The data analyzing module 1322 receives and analyzes the illumination state information, the parking state information, the tag analysis information, and the tag identification information from the network interface 1320 to create movement information, parking time information, and parking charge information of a vehicle. In other words, if sequentially receiving tag identification information from the tag-ZigBee device 1150, the data analyzing module 1322 matches the tag identification information with tag analysis information to identify the vehicle, so that the movement information of the vehicle can be created based on information about coordinates of the second ZigBee device 1255.

The controller 1326 forms a screen image based on map information of a parking system, information about coordinates of the second ZigBee device 1255, and mapping information, and forms a management data image by applying illumination state information, parking state information, vehicle information, movement information of the vehicle, parking time information of the vehicle, and parking charge information of the vehicle to the screen image.

In addition, when receiving parking completion information (representing that a vehicle is parked) from the tag-ZigBee device 1150 through the second ZigBee device 1255, the data analyzing module 1322 calculates parking time from a time point in which the parking completion information is received. Thereafter, when the vehicle moves or charge settlement for parking is requested by the parking information guiding device 1310, the data analyzing module 1322 can create parking charge information.

The controller 1326 controls the operation of each element. The controller 1326 can extract and transmit desired information according to the request of the parking information guiding device 1310. The information extracted by the controller 1326 is delivered to the parking information guiding device 1310 through the network interface 1320. The network interface 1320 can be connected with the parking information guiding device 1310 over a cable network comprising a UART, a universal serial bus (USB), a serial/parallel cable, and the Internet (TCP/IP).

The user interface 1330 displays the management image data and provides the management image data to a manager. Since the management image data can be formed through various formats, the manager can check only necessary information or manage the management image data in various formats by manipulating the user interface 1330. In addition, the user interface 1330 may comprise manipulation units such as a touch screen, a button, and a keyboard.

The D/B managing module 1324 records information transmitted in the data analyzing module 1322 and the controller 1326 in the D/B 1328, and controls inputting/outputting and deleting information.

The D/B 1328 stores map information, coordinate information, and mapping information, and has a program to apply the information to illumination state information, parking state information, movement information of a vehicle, and tag analysis information. The program can be executed by the controller 1326.

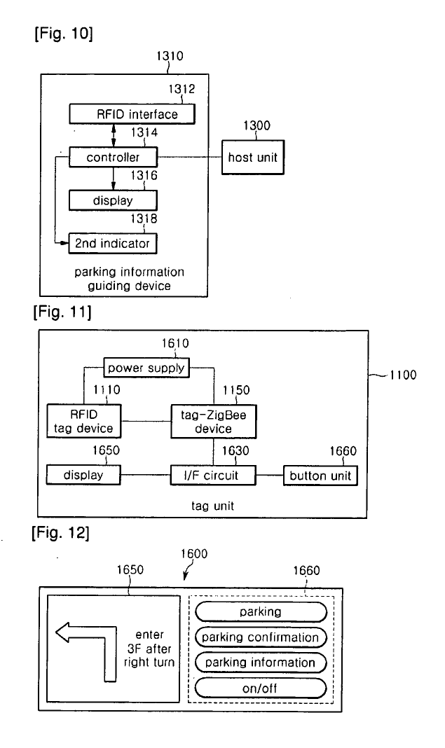

FIG. 10 is a control block diagram showing the parking information guiding device 1310 of the management system of the public facilities according to the first embodiment of the present invention.

The parking information guiding device 1310 comprises an RFID interface 1312, a controller 1314, a display 1316, and a second indicator 1318.

The RFID interface 1312 may serve as an RF reader such as the RFID reader device 1210. When a driver allows the tag unit 1100 to be recognized, the RFID interface 1312 analyzes tag identification information to create tag analysis information.

If receiving tag analysis information from the RFID interface 1312, the controller 1314 regards parking information as requested by a driver and requests data to the host unit 1300.

At this time, the host unit 1300 extracts parking state information (such as information about a parking region in which a corresponding vehicle is parked), parking time information, and parking charge information to form management image data based on the information. The host unit 1300 transmits the management image data to the parking information guiding device 1310.

The parking information guiding device 1310 can calculate final charges by reflecting a time point, in which a driver allows the tag unit 1100 to be recognized, on the parking charge information and then transmit the parking charge information to the host unit 1300. Such the parking charge information is recorded in the DB 1328 of the host unit 1300.

The parking information guiding device 1310 can display the management image data on the display 1316 and guide a position in which a vehicle is parked.

The second indicator 1318 may comprise an LED or an alarm lamp. The second indicator 1318 is turned on by a control operation of the controller 1314 if the tag unit 1100 enters a coverage area of the RFID interface 1312. The second indicator 1318 is turned off by the control operation of the controller 1314 if the tag unit 1100 deviates from the coverage area. In this case, when the tag unit 1100 enters the coverage area, the second indicator 1318 may blink in order to attract the attention of a driver.

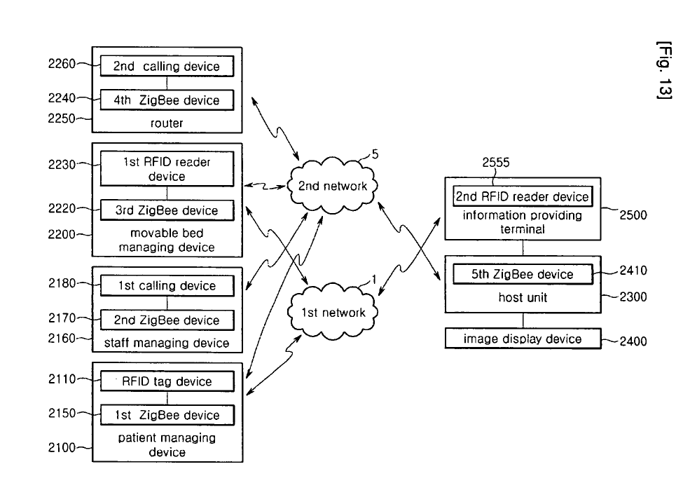

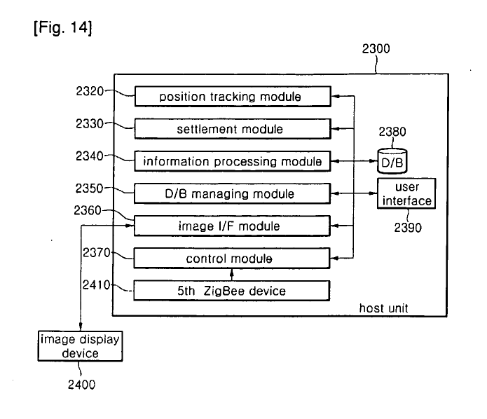

FIG. 11 is a control block diagram showing the tag unit 1100 of the management system of the public facilities according to the first embodiment of the present invention, and FIG. 12 is a view showing an example of a guide image provided by the tag unit 1100 of the management system of the public facilities according to the first embodiment of the present invention.

Referring to FIG. 11, the tag unit 1100 comprises a power supply 1610, the RFID tag device 1110, the tag-ZigBee device 1150, a display 1650, an interface (I/F) circuit 1630, and a button unit 1660.

The power supply 1610 supplies power to all circuits such as the display 1650, the I/F circuit 1630, and the button unit 1660.

The tag-ZigBee device 1150 requests its data related to parking to the host unit 1300, and the host unit 1300 extracts the data. The data may comprise tag analysis information, coordinate information, parking state information, parking time information, and parking charge information.

The host unit 1300 transmits the extracted data to the tag-ZigBee device 1150 through the third ZigBee device 1340, and a processor of the tag-ZigBee device 1150 forms guide information by using the data.

A driver can select and input a command by manipulating the button unit 1660, and the I/F circuit 1630 converts a key input signal of the button unit 1660 into an interrupt signal to deliver the key input signal to the tag-ZigBee device 1150.

If a person presses a parking confirmation button, the tag-ZigBee device 1150 confirms the parking state information matched with coordinate information to detect a present position of a person who has left a vehicle through the host unit 1300.

The tag-ZigBee device 1150 confirms the parking state information based on the coordinate information to create guide information. As person possessing the tag unit 1100 moves, the tag-ZigBee device 1150 recognizes a present position in real time through the host unit 1300 to display guide information.

FIG. 12 shows a guide image 1600. In the button unit 1660 of the guide image 1600, a parking button is used to search for an available parking region, and a parking information button is used to check parking time and parking charge information. In addition, a user can manually control the power supply 1610 by pressing an on/off button without selecting modes.

FIG. 13 is a control block diagram showing a management system of public facilities according to a second embodiment of the present invention, and, particularly, shows a case in which the present invention is applied to a hospital managing system.

As shown in FIG. 13, the hospital managing system according to an embodiment comprises a patient managing device 2100 equipped with a first ZigBee device 2150 and an RFID tag device 2110, a router 2250 equipped with a fourth ZigBee device 2240 and a second calling device 2260, a host unit 2300 equipped with a fifth ZigBee device 2410, a staff managing device 2160 equipped with a second ZigBee device 2170 and a first calling device 2180, a movable bed managing device 2200 equipped with a first RFID reader device 2230 and a third ZigBee device 2220, an information providing terminal 2500 equipped with a second RFID reader device 2555, and an image display device 2400.

The RFID tag device 2110, the first RFID reader device 2230, and the second RFID reader device 2555 make RFID communication through the first network 1. The first ZigBee device 2150, the second ZigBee device 2170, the third ZigBee device 2220, the fourth ZigBee device 2240, and the fifth ZigBee device 2410 make ZigBee communication through the second network 5.

The patient managing device 2100 is provided to a patient when information about the patient is received in the hospital managing system, and is a portable tag-type device. The patient managing device 2100 comprises the RFID tag device 2110 transmitting/receiving data through the first network 1 and the first ZigBee device 2150 transmitting/receiving data through the second network 5.

The RFID tag device 2110 transmits patient information with tag identification information. Such an RFID tag device 2110 is a passive tag. The RFID tag device 2110 can transfer/modify information based on new information only by nearing a reader. Accordingly, when the patient managing device 2100 is provided to a patient, information about the patient can be easily recorded in the RFID tag device 2110.

The first ZigBee device 2150 transmits/receives patient information through the second network 5 to tract a position of a patient. The first ZigBee device 2150 can communicate with the host unit 2300 through the fourth ZigBee device 2240 serving as a router.

The staff managing device 2160 is provided to persons (such as a nurse and a doctor) concerned in a hospital, and is a portable tag-type device. The staff managing device 2160 comprises the second ZigBee device 2170 transmitting/receiving data through the second network 5 and the first calling device 2180 capable of displaying data.

The second ZigBee device 2170 transmits ZigBee identification information and staff information. The second ZigBee device 2170 can communicate with the host unit 2300 through the fourth ZigBee device 2240 serving as a router. Accordingly, the host unit 2300 can tract the position of a staff member, and can transmit patient transport information and staff call information to the second ZigBee device 2170.

The first calling device 2180 displays the patient transport information and the staff call information received through the second ZigBee device 2170. The first calling device 2180 may be realized by using an audio device and a liquid crystal display (LCD). The first calling device 2180 may be manufactured in a small size, and may be a portable device like the second ZigBee device 2170.

Differently from the patient managing device 2100, the staff managing device 2160 having the above structure can be durably used if the staff managing device 2160 is once provided to staffs. Therefore, information of the staff managing device 2160 is necessary to be upgraded. Accordingly, if staff information is necessary to be upgraded, the second ZigBee device 2170 is connected to the host unit 2300 through a cable transmission technology related to a USB and an UART to update the staff information.

The movable bed managing device 2200 is attached to a movable bed to carry a patient when the patient receives medical treatment or goes through an operation. Accordingly, the movable bed managing device 2200 comprises the first RFID reader device 2230 receiving patient information from the RFID tag device 2110 of the patient managing device 2100 possessed by a patient and the third ZigBee device 2220 transmitting the patient information to the second network 5. Therefore, the host unit 2300 can tract information about the patient on a movable bed and a place to which the patient bed moves.

The router 2250 comprises the fourth ZigBee device 2250 routing data among the RFID tag device 2110, the staff managing device 2160, the movable bed managing device 2200, and the host unit 2300, and the second calling device 2260 displaying data

Fourth ZigBee devices 2240 of the router 2250 are regularly arranged on ceilings of corridors and a variety of rooms, such a treatment room, a test room, a sickroom, a medical consultation room, a drug room, a material room, a rest room, and a refectory. Accordingly, the host unit 2300 can obtain coordinates of installation areas of the fourth ZigBee devices 2240 and applies the coordinates on a map. In addition, the host unit 2300 can trace positions of ZigBee devices communicating with the fourth ZigBee devices 2240 according to installation information of the fourth ZigBee devices 2240. Accordingly, the host unit 2300 can manage the patient managing device 2100, the staff managing device 2160 or the movable bed managing device 2200 communicating with the fourth ZigBee devices 2240 by tracking the position of the patient managing device 2100, the staff managing device 2160 or the movable bed managing device 2200.

The second calling device 2260 displays patient transfer information and staff call information received in the fourth ZigBee device 2240. Accordingly, the router 2250 receives patient transfer information from the host unit 2300 installed in a laboratory or an operating room and notifies the patient transfer information through the second calling device 2260, so that persons concerned in the hospital can prepare for consultation or operation. The second calling device 2260 signal-processes information received in the fourth ZigBee device 2240 to form voice data, text data, or image data and convert the data into driving signals. The patient transfer information, the staff calling information, and the patient handling information converted into the driving signals can be provided through a speaker, an electric bulletin, an LCD, and an alarm.

The fifth ZigBee device 2410 of the host unit 2300 serves as a coordinator to manage the second network 5, and transmits/receives various information after collecting the information, gathering statistics related to the information, processing the information, and settling charges related to the information. Accordingly, the host unit 2300 may be installed in a monitoring room, a reception desk, and a center in a hospital to manage positions of patients and staff, guide medial consultation service, settle an account, and make a schedule for medial consultation.

The host unit 2300 is connected to the information providing terminal 2500 and the image display device 2400 to display various data.

For example, the information providing terminal 2500 may be installed in almost all parts of the hospital in the form of a KIOSK. The information providing terminal 2500 receives data from the host unit 2300 to guide medial consultation, hospital facilities, an information reception service to clients.

The information providing terminal 2500 comprises the second RFID reader device 2555. The information providing terminal 2500 communicates with the RFID tag device 2110 of the patient managing device 2100 to identify a patient. Accordingly, the information providing terminal 2500 can provide a service required by the patient.

The image display device 2400 is a kind of an electric bulletin installed in a reception desk, a medical consultation room, a consultation room, a treatment room, and a sickroom. The image display device 2400 receives corresponding data from the host unit 2300 to guide a medial consultation order, examinee information, medical consultation schedule information, and information about the present status of patients.

Through the above structure, a staff member in a hospital wears the staff managing device 2160, and a patient, who has gone through a reception procedure in a reception desk, receives and wears the patient managing device 2100. The staff managing device 2160 and the patient managing device 2100 may have the form of a card.

In the case of the patient managing device 2100, information about a new patient can be recorded by a reader when the patient managing device 2100 is provided, and the host unit 2300 registers identification information of the RFID tag device 2110 and patient information matched with the identification information of the RFID tag device 2110 in a database. Thereafter, when the patient visits a hospital again, a manager determines that information about the patient is registered in the host unit 2300, and searches for the patient managing device 2100 corresponding to the patient to provide the patient managing device 2100 to the patient.

The first ZigBee device 2150 of the patient managing device 2100 communicates with the fourth ZigBee device 2240 of the router 2250 installed in each region, and the fourth ZigBee device 2240 transmits patient information of the first ZigBee device 2150 to the host unit 2300. The host unit 2300 can track the position of a patient based on coordinate information of the fourth ZigBee device 2240 of the host unit 2300. For example, a staff member determines if a patient is in a restricted region, enters a sickroom, or is at another place in a medical consultation time to manage the patient.

When a patient places the patient managing device 2100 near the information providing terminal 2500, the patient managing device 2100 makes RFID communication with the information providing terminal 2500. After identifying the patient managing device 2100 based on tag identification information, the second RFID reader device 2555 receives corresponding patient information from the host unit 2300. The information providing terminal 2500 can provide a user interface such as a touch screen to allow a patient to input desired service, and can request desired data to the host unit 2300.

Accordingly, the information providing terminal 2500 can provide consultation guide information (e.g., consultation hours, a schedule of a consultant, consultation order, and waiting time), facility guide information (e.g., a type, a position, and use time of facilities in a hospital), parking guide information (e.g., a position of a parking lot, a present parking state, a parking time, and parking charge settlement), settlement guide information related to reception of charges for consultation, and prescription guide information by using an image.

Meanwhile, when a patient lies down on a movable bed, the RFID tag device 2110 of the patient managing device 2100 transmits patient information to the first RFID reader device 2230 of the movable bed managing device 2200. The third ZigBee device 2220 transmits the patient information and identification information of a first reader to the host unit 2300 through the fourth ZigBee device 2240. Accordingly, the host unit 2300 can detect a movable bed used by a specific patient and can trace the position of the patient by detecting the moving route of the movable bed.

The host unit 2300 can determine a medical consultation/treatment schedule of a patient to indicate preparation for consultation/operation/test according to a present position of a patient to the second calling device 2260 of the router 2250.

In addition, the host unit 2300 transmits patient transfer information, staff calling information, and patient handling information to the router 2250 through the second network 5, and the router 2250 converts the information received in the second calling device 2260 into multimedia data.

FIG. 14 is a control block diagram showing the host unit 2300 of the management system of the public facilities according to the second embodiment of the present invention.

The host unit 2300 comprises a position tracking module 2320, a settlement module 2330, an information processing module 2340, a D/B managing module 2350, an image I/F module 2360, a controller 2370, a D/B 2380, a user interface 2390, and a fifth ZigBee device 2410.

The database 2380 stores reception information of a patient, coordinate information of the router 2250, map information of a hospital, matching information, consultation guide information, facility guide information, parking guide information, settlement guide information, prescription guide information, patient transfer information, staff calling information, patient handling information, information about the present status of patients in a sickroom, and staff/patient position information.

If information of the router 2250 is received, the position tracking module 2320 identifies the patient managing device 2100, the staff managing device 2160, and the movable bed managing device 2200 which have communicated with the router 2250 to generate position information of a corresponding device based on matching information, map information, and coordinate information of the router 2250 such that position information of a corresponding device is generated. Such the position information is periodically created according to a period for transmitting information of the router 2250 and stored in the D/B 2380 in real time. Accordingly, the position information recorded in the D/B 2380 may be utilized as movement information of a patient or a staff member.

The settlement module 2330 compares the reception information of a patient with the position information of the patient. The settlement module 2330 determines if the patient actually undergoes medical consultation/treatment to create settlement information.

The information processing module 2340 extracts information from the D/B 2380 and forms a data packet according to a destination in which the information is provided to the second calling device 2260 of the router 2250, the first calling device 2180 of the staff managing device 2160, the information providing terminal 2500, and the image display device 2400. The data packet can be transmitted to the staff managing device 2160, the router 2250, the information providing terminal 2500, and the image display device 2400 through the fifth ZigBee device 2410 or a cable network.

For example, consultation guide information, facility guide information, parking guide information, settlement guide information, and prescription guide information may be transmitted to the information providing terminal 2500 through the cable network. In addition, consultation guide information, prescription guide information, and information about a present state of patients in a sickroom may be transmitted to the image display device 2400. In addition, staff calling information, patient transfer information, and patient handling information may be transmitted to the second calling device 2260 of the router 2250 or the first calling device 2180 of the staff managing device 2160 through the second network 5.

The user interface 2390 modifies data recorded in the D/B 2380, or selects the type of data transmitted to the staff managing device 2160, the router 2250, the information providing terminal 2500, and the image display device 2400. The user interface 2390 provides a screen image for information input when information related to hospital operation and various guide information are modified, and input data are transmitted to the D/B managing module 2350 and a module to process corresponding algorithm. The user interface 2390 may comprise a manipulation such as a touch screen, a button, and a key board.

When each module requests data, the D/B managing module 2350 controls data input/output of the D/B 2380, and records/modifies/delete data.

The image I/F module 2360 forms a format of data to be provided to the image display device 2400 such that format satisfies an image standard, and the data is transmitted a driving circuit of the image display device 2400.

The control module 2370 is connected to the fifth ZigBee device 2410 to convert ZigBee data received from the fifth ZigBee device 2410 into digital data which can be analyzed and transmit the converted data to the modules. In addition, the control module 2370 comprises a kernel to interfere with the modules, processes an interrupt, and operates the above modules by providing request processing time and operation priority.

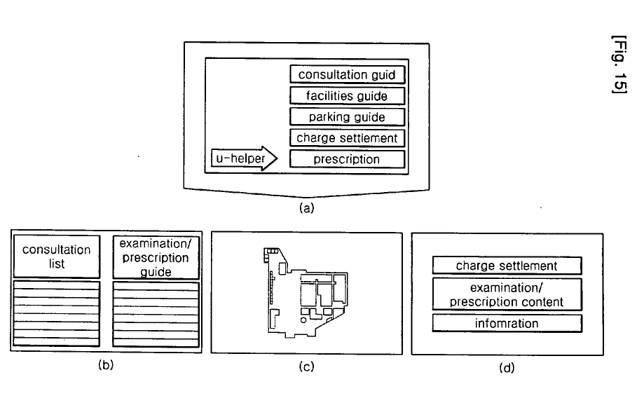

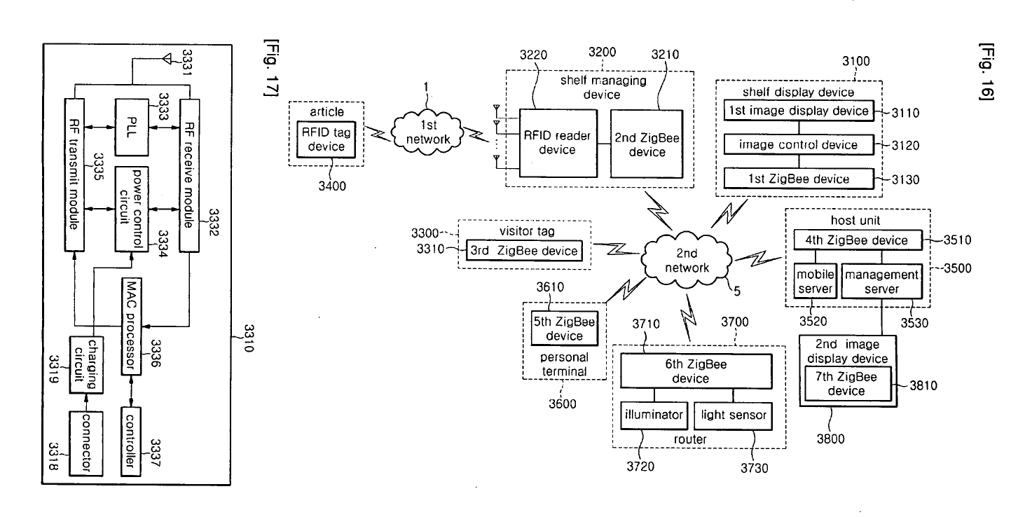

FIGS. 15A to 15D are views showing images displayed by the information providing terminal 2500 of the management system of the public facilities according to the second embodiment of the present invention.

FIG. 15A shows a touch screen image when a patient connects the patient managing device 2100 with the information providing terminal 2500, and FIG. 15B shows an image providing consultation guide information and prescription guide information.

In addition, FIG. 15C shows facility guide information displayed by the information providing terminal 2500, and FIG. 15D shows an image guiding printing of settlement information and prescription guide information.

FIG. 16 is a control block diagram showing a management system of public facilities according to a third embodiment of the present invention, and, particularly, shows a case in which the present invention is applied to a store managing system.

As shown in FIG. 16, the store managing system comprises a visitor tag 3300 and an RFID tag device 3400 attached to an article. The store managing system comprises a shelf display device 3100 processing and transmitting identification information received from the visitor tag 3300 and the RFID tag device 3400, a shelf managing device 3200, and a router 3700. The store managing system comprises a router 3700, a host unit 3500 managing the movement of a visitor's article provided in a store by processing identification information and providing additional information such as sale information to a visitor, a personal terminal 3600 displaying data, and a second image display device 3800.

The visitor tag 3300 is a card-type tag. The visitor tag 3300 is mounted on a cart or provided to a customer when the customer enters into a store. The visitor tag 3300 comprises a third ZigBee device 3310 transmitting/receiving data through the second network 5 which is a ZigBee network. The third ZigBee device 3310 transmits appliance identification information to a second ZigBee device 3210 of the shelf managing device 3200 and a six ZigBee device 3710 of the router 3700. The third ZigBee device 3310 can receive power through a rechargeable connector.

An RFID tag device 3400 is a tag attached to an article, and comprises identification information of the article.

A plurality of shelf managing devices 3200 are provided to manage a plurality of shelves by dividing the shelves into several group units. The shelf managing device 3200 comprises an RFID reader device 3220 receiving tag identification information of the RFID tag device 3400 through the first network 1 and the second ZigBee device 3210 transmitting/receiving data through the second network 5.

The shelf display device 3100 is a kind of an electronic shelf label (ESL). A plurality of shelf display devices 3100 are installed according to types of articles arranged on shelves. The shelf display device 3100 displays article information, advertisement information, sale information, discount information, information for sale based on order of arrival, customer position information, and bulletin information. The shelf display device 3100 comprises a first image display device 3110, an image control device 3120, and a first ZigBee device 3130.

The host unit 3500 comprises a fourth ZigBee device 3510, a mobile server 3520, and a management server 3530.

The personal terminal 3600 comprises a fifth ZigBee device 3610, and the router 3700 comprises a sixth ZigBee device 3710, an illuminator 3720, and a light sensor 3730.

The second image display device 3800 is connected to the host unit 3500, and comprises a seventh ZigBee device 3810.

The RFID tag device 3400 and the RFID reader device 3220 transmits/receives tag identification information to/from each other through the first network 1 having an RFID channel.

The first ZigBee device 3130 to the sixth ZigBee device 3710 make data communication through the second network 5 having a ZigBee channel. In this case, the first ZigBee device 3130 of the shelf display device 3100, the third ZigBee device 3310 of the visitor tag 3300, and the fifth ZigBee device 3610 of the personal terminal 3600 may comprise an RFD. The second ZigBee device 3210 of the shelf managing device 3200 and the sixth ZigBee device 3710 of the router 3700 comprise an FFD capable of performing a routing function. The fourth ZigBee device 3510 of the host unit 3500 serves as a PAN coordinator.

Through the above structure, when articles are arranged on shelves or when a customer selects the articles to contain the articles in a cart, the RFID tag device 3400 of the article transmits tag identification information to the RFID reader device 3220 of the shelf managing device 3200.

The shelf managing device 3200 receives article information from the RFID tag device 3400 and transmits the article information to the shelf display device 3100 and the host unit 3500.

Accordingly, the shelf display device 3100 can display information about articles arranged on shelves.

The host unit 3500 can create arrangement information about articles on the shelves when RFID tag devices 3400 (articles) are arranged on the shelves.

In contrast, the host unit 3500 can create movement information about articles by tracking a position of the second ZigBee device 3210 of the shelf managing device 3200 when the RFID tag devices 3400 are contained in a cart.

The shelf managing device 3200 transmits a signal of the visitor tag 3300 to the fourth ZigBee device 3510 of the host unit 3500, and the host unit 3500 receives the signal of the visitor tag 3300 to track the position of the second ZigBee device 3210, thereby creating customer position information.

The shelf managing device 3200 receives article information, advertisement information, sale information, discount information, information for sale based on order of arrival, customer position information, bulletin information, movement information about articles from the host unit 3500 and transmits the information to the shelf display device 3100, so that the shelf display device 3100 can display the information. At this time, the fourth ZigBee device 3510, the second ZigBee device 3210, and the first ZigBee device 3130 communicate with each other.

As described above, the second ZigBee device 3210 of the shelf managing device 3200 performs a router function with the sixth ZigBee device 3710, and communicates with the first ZigBee device 3130, the third ZigBee device 3310, the fourth ZigBee device 3510, and the fifth ZigBee device 3610.

The sixth ZigBee device 3710 performs a routing function used to construct a ZigBee network, and communicates with the third to fifth ZigBee devices 3310, 3610, and 3510 of the visitor tag 3300, the personal terminal 3600, and the host unit 3500. According to the present embodiment, the router 3700 is installed with illuminators 3720 uniformly arranged on a ceiling of a store. Accordingly, the host unit 3500 represents the arrangement of the illuminators 3720 in the form of coordinates and analyzes the position of the router 3700 installed with the illuminator 3720 to track the position of the visitor tag 3300 communicating with the sixth ZigBee device 3710 of the router 3700.

The fourth ZigBee device 3510 of the host unit 3500 communicates with the second

ZigBee device 3210 and the six ZigBee device 3710 of the shelf managing device 3200 and the router 3700 to collect various information. Accordingly, the host unit 3500 tracks the position of the shelf managing device 3200 or the router 3700 which has transmitted appliance identification information of the visitor tag 3300 to create customer position information. The customer position information comprises information about a present position of a customer and information about a moving path of the customer.

The management server 3530 of the host unit 3500 is a computer to manage a store by collecting transmitted/received information, gathering statistics related to the information, and settling charges related to the information. The management server 3530 processes article information, advertisement information, sale information, discount information, information for sale based on order of arrival, customer position information, movement information of articles, settlement information, customer preference information, coordinate information of the sixth ZigBee device 3710, map information, article arrangement information, and stored article information.

The mobile server 3520 of the host unit 3500 transmits bulletin information requested through the personal terminal 3600 to the first ZigBee device 3130, so that the first image display device 3110 can be utilized as a bulletin.

The personal terminal 3600 is a mobile communication terminal such as a portable phone, a smart phone, and a personal digital assistant (PDA) phone belonging to a customer visiting in a store. The personal terminal 3600 is not installed with the store managing system according to an embodiment. However, if the personal terminal 3600 comprises a ZigBee communication device, that is, the fifth ZigBee device 3610, the personal terminal 3600 can be connected to the second network 5 to process various information. Therefore, according to the embodiment, the personal terminal 3600 may be an element of the store managing system.

In this case, a store manager can provide a service such that a customer can use the shelf display device 3100 as a personal bulletin board. Bulletin information about the bulletin board refers to multimedia information created through manipulation of the personal terminal 3600 by the customer.

If the customer requests a personal bulletin service, the store manager receives a second connection number used to connect to the mobile server 3520 of the host unit 3500. Then, after the personal terminal 3600 of the customer is connected to the mobile server 3520 through the second connection number, the bulletin information can be transmitted.

When the customer requests the personal bulletin service, the store manager registers network information of the personal terminal 3600 in the host unit 3500, and the customer can transmit the bulletin information to the shelf display device 3100 through the personal terminal 3600.

In addition, the customer creates multimedia data by manipulating the personal terminal 3600, and can form bulletin information by using the multimedia data. The bulletin information is transmitted to the first ZigBee device 3130 of the shelf display device 3100 through the fifth ZigBee device 3610 of the personal terminal 3600, so that the shelf display device 3100 can display the bulletin information.

In other words, the fifth ZigBee device 3610 of the personal terminal 3600 can transmit bulletin information to the first ZigBee device 3130 through the sixth ZigBee device 3710 or the second ZigBee device 3210. The personal information is collected in the mobile server 3520 through a mobile communication network, processed, and then transmitted to the shelf display device 3100 through the fourth ZigBee device 3510.

Similarly to the third ZigBee device 3310 of the visitor tag 3300, the fifth ZigBee device 3610 of the personal terminal 3600 communicates with the second ZigBee device 3210 or the sixth ZigBee device 3710 to track a position thereof. The fifth ZigBee device 3610 can receive power from a battery of the personal terminal 3600.

Second image display devices 3800 are installed on corridors of a store and a guide desk to guide a position of a missing child, information about a present position of a customer, and map information of a store. The second image display devices 3800 may be realized in the form of a KIOSK. The second image display devices 3800 is connected to the management server 3530 by using a cable to receive guide information from the management server 3530, and transmit appliance identification information and customer information of the third ZigBee device 3310 or the fifth ZigBee device 3610 to the management server 3530.

A customer allows the third ZigBee device 3310 of the visitor tag 3300 or the fifth ZigBee device 3610 of the personal terminal 3600 to be recognized by the seventh ZigBee device 3810 of the second image display device 3800, and can register customer information through an interface unit such as a touch screen.

Through the above structure, a customer visiting in a store receives the visitor tag 3300 or obtains a cart having the visitor tag 3300. If the customer visits in the store with a child, the child additionally receives the visitor tag 3300 preventing the child from being lost to wear the visitor tag 3300 in the form of a card, a necklace, and a bracelet. When the visitor tag 3300 is provided, customer information matched with appliance identification information of the visitor tag 3300 may be registered in the management server 3530. In addition, as described above, appliance identification information and customer information may be registered in the management server 3530 through the seventh ZigBee device 3810 of the second image display device 3800. The customer information may be registered in the management server 3530 by a person providing the visitor tag 3300 (e.g., store manager). The customer information comprises personal information (e.g., a name of a customer, an address of a place of residence, and a phone number).

When articles are arranged, the RFID tag device 3400 transmits article information and appliance identification information to the RFID reader device 3220 of the shelf managing device 3200, and the RFID reader device 3220 analyzes the information. The appliance identification information of the RFID tag device 3400 may comprise hardware information such as a MAC address, and the article information may comprise a type, a price, a manufacturer, and an expiration date of a produce.

The article information having an RFID data format is analyzed into digital data having a format employed in the host unit 3500. The article information is transmitted to the first ZigBee device 3130 of the shelf display device 3100 and the fourth ZigBee device 3510 of the host unit 3500 through the second ZigBee device 3210 of the shelf managing device 3200.

The article information transmitted to the shelf display device 3100 is used to form multimedia data in the image control device 3120, and the first image display device 3110 displays article information comprising the multimedia data.

The article information transmitted to the host unit 3500 is transmitted to the management server 3530, and the management server 3530 modifies arrangement information about articles by taking the article information into consideration. The RFID reader device 3220 has been installed on a shelf, and the management server 3530 identifies the shelf according to a reader identification number of the RFID reader device 3220. Accordingly, the management server 3530 can recognize information about shelves and information about articles on the shelves.

When a customer selects articles and puts the articles into a cart, the RFID tag device 3400 transmits article information to the RFID reader device 3220 of the shelf management device 3200, and the article information received in the RFID reader device 3220 is transmitted to the fourth ZigBee device 3510 of the host unit 3500 through the second ZigBee device 3210. In this case, article information transmitted from the second ZigBee device 3210 may be transmitted to the host unit 3500 through the router 3700.

The management server 3530 of the host unit 3500 marks coordinates of the shelf managing device 3200 transmitting article information on a map to create movement information of articles. The host unit 3500 maps the position of the shelf managing device 3200 transmitting article information with the position of the shelf managing device 3200 transmitting appliance identification information of the visitor tag 3300 so that information about an article purchased by a customer is analyzed. Accordingly, the management server 3530 can process customer position information, movement information of articles, settlement information based on article information, article arrangement information (comprising information about the number of articles on shelves), stored article information, and customer preference information.

For example, customer preference information may comprise information about an article selected by a customer, a shopping route according to a movement of the customer, a movement time (which may be calculated based on difference in transmission time for article information from various second ZigBee devices 3210 or various sixth ZigBee devices 3710), and time (purchase determination time) taken to purchase an article by the customer. According, the management server 3520 can provide customer relationship management (CRM) service to the store manager based on the customer preference information.

Within a predetermined time after movement information of an article is created, that is, after an article disappears, if charges for the article are not paid on a cash desk, the management server 3530 may regard the article as stolen.

If the management server 3530 matches movement information of a customer with the movement information of the article, so that the customer goes out of the store, but the matched article is not paid, the management server 3530 may regard the article as stolen. Accordingly, the management server 3530 can trace customer information to determine a customer stealing the article.

Meanwhile, a customer may grip an article in order to select the article before putting the article into a cart. In this case, the shelf management device 3200 may detect this.

At this time, the RFID reader device 3220 comprising a plurality of antennas mounted on several areas of shelves identifies an antenna receiving article information of the RFID tag device 3400 to create movement information of the article when the customer grips the article to change the position of the article. The movement information of the article is created by detecting small position variation of the article generated from a small area around a shelf of the article. In other words, even if the customer observes the article carefully and then puts down the article at its original position or adjacent another position, the movement information of the article may be created.

The RFID reader device 3220 creates movement information of an article based on an antenna in real time, and the movement information of the article is transmitted to the management server 3530 of the host unit 3500 through the second ZigBee device 3210.

In this case, the management server 3530 calculates time taken to receive the movement information of the article based on the antenna in real time, and time in which the customer observes the article.

When the movement information of the article is created, the management server 3530 determines if the article is an event article and transmits advertisement information, sale information, and discount information of the article to the first ZigBee device 3130 of the shelf display device 3100. Accordingly, the shelf display device 3100 can supply purchase information guiding service to a customer observing the article in real time.

If the customer, who has checked advertisement information, sale information, and discount information of the article, puts the article into the cart, the shelf management device 3200 or the router 3700 transmits the article information to the management server 3530. The management server 3530 creates information for sale based on order of arrival according to time sequence as information of articles is received therein. The information for sale based on order of arrival may serve as a criterion of a discount rate of articles when settlement information of the articles is created.

Meanwhile, when a customer loses a child, the visitor tag 3300 of the child communicates with the shelf management device 3200 or the router 3700, so that the position of the child can be traced.

The management server 3530 creates customer position information of the child and transmits the customer position information to the second image display device 3800 or to the shelf display device 3100 through the fourth ZigBee device 3510. In this case, since the second image display device 3800 or the shelf display device 3100 has map information and mapping information, the second image display device 3800 or the shelf display device 3100 marks the customer position information of the child on a map and display the map.

According to another embodiment, various services can be provided through the personal terminal 3600.

Similarly to the personal terminal 3600, the mobile server 3520 of the host unit 3500 comprises an RF transmit module, a base-band module, and an operation module. In addition, since the mobile server 3520 of the host unit 3500 has a communication protocol, the mobile server 3520 of the host unit 3500 serves as a computer communicating with a base station of a service provider.

When a customer enters in a store, the customer receives a connection number capable of accessing the mobile server 3520. Accordingly, when the customer wants to recognize the position of his/her lost child or use the shelf display device 3100 as a personal bulletin board, the customer can access the mobile server 3520 with the connection number.

For example, the connection number may be classified into a first connection number used to search for a lost child and a second connection number used to use a personal bulletin board service.

If the customer accesses the mobile server 3520 through the personal terminal 3600 by using the first connection number and transmits customer information of the lost child through a short message service (SMS), the mobile server 3520 transmits the customer information to the management server 3530 while requesting customer position information of the lost child to the management server 3530. Accordingly, the management server 3530 confirms the customer position information of the lost child matched with customer information registered when the lost child enters the store, and transmits the customer position information to the mobile server 3520. The mobile server 3520 makes an SMS message based on the customer position information of the lost child and transmits the SMS message to the personal terminal 3600. Accordingly, the customer checks the SMS message to recognize the position of his/her lost child.

Meanwhile, when a customer accesses the mobile server 3520 through the personal terminal 3600 by using the second connection number and transmits multimedia data (characters, still image, moving pictures, etc.) for a personal bulletin board, the mobile server 3520 transmits the multimedia data to the management server 3530 while requesting the activation of the personal bulletin board.

The multimedia data received in the management server 3530 is transmitted to the first ZigBee device 3130 through the fourth ZigBee device 3510 and the second ZigBee device 3210 or the sixth ZigBee device 3710, and the image control device 3120 processes the multimedia data and transmits the multimedia data to the first image display device 3110. Accordingly, the first image display device 3110 displays the multimedia data suitable for the size of a screen.

The personal bulletin board function may be performed through the second network 5 without the mobile server 3520, that is, mobile communication.

If a customer manipulates the personal terminal 3600 and commands the personal terminal 3600 to transmit multimedia data, the fifth ZigBee device 3610 converts the multimedia data into ZigBee data and transmits the multimedia data to the second ZigBee device 3210 or the sixth ZigBee device 3710. The multimedia data received in the second ZigBee device 3210 or the sixth ZigBee device 3710 are transmitted to the first ZigBee device 3130, and displayed through the first image display device 3110 as described above.

Further, in order to attract an attention of a customer, the shelf display device 3100 extracts advertisement information, sale information, and discount information of articles arranged on the shelves to randomly display the information with article information of the articles.

If new information of articles is generated, the management server 3530 creates upgrade information to transmit the upgrade information through the shelf display device 3100 through the fourth ZigBee device 3510, the second ZigBee device 3210 (or the sixth ZigBee device 3710), and the first ZigBee device 3130. When receiving the upgrade information, the image control device 3120 upgrades advertisement information, sale information, and discount information and displays upgrade information.

The management server 3530 provides an illumination management screen image/through a user interface, and a manager can control the illuminator 3720 by manipulating the illumination management screen image.

When receiving illumination control information, the management server 3530 transmits the illumination control information to the fourth ZigBee device 3510, and the fourth ZigBee device 3510 transmits the control information to the sixth ZigBee device 3710. The sixth ZigBee device 3710 controls a driving circuit or power according to the illumination control information such that the illuminator 3720 is turned on/off or dimmed.

The illuminator 3720 may comprise various devices such as a fluorescent lamp or an LED. The light sensor 3730 is mounted on a ceiling and connected to the sixth ZigBee device 3710. The light sensor 3730 transmits information about quantity of detected light to the sixth ZigBee device 3710, and the sixth ZigBee device 3710 can control the operation of the illuminator 3720 according to illumination conditions.

The sixth ZigBee device 3710 transmits information about the operation of the illuminator 3720 to the host unit 3500, and the host unit 3500 maps coordinate information of the sixth ZigBee device 3710 with map information. The host unit 3500 marks the operation information of the illuminator 3720 on map information to provide the operation information of the illuminator 3720 through a user interface, so that a manager can monitor an illumination state of a store.

FIG. 17 is a control block diagram showing the third ZigBee device of the management system of the public facilities according to the third embodiment of the present invention.

The third ZigBee device 3310 of the visitor tag 330 comprises an antenna 3331, an RF receive module 3332, an RF transmit module 3335, a PLL 3333, a power control circuit 3334, a MAC processor 3336, a controller 3337, a connector 3318, and a charging circuit 3319. Since the third ZigBee device 3310 has the same operation and structure as those of the ZigBee device 230 (see FIG. 7), details thereof will be omitted in order to avoid redundancy.

The connector 3318 has a shape similar to that of a charging port of a mobile phone. The connector 3310 may be realized by using various connection devices such as a 24-pin connector.

The charging circuit 3319 comprises a rechargeable battery, a charge state detector, and a voltage stabilizing circuit. The charging circuit 3310 transmits charged power to the power control circuit 3334.

For the purpose of convenience of a customer, battery chargers for a portable phone are disposed in many places of a store. The customer can easily charge the visitor tag 3300 by using the battery charger.

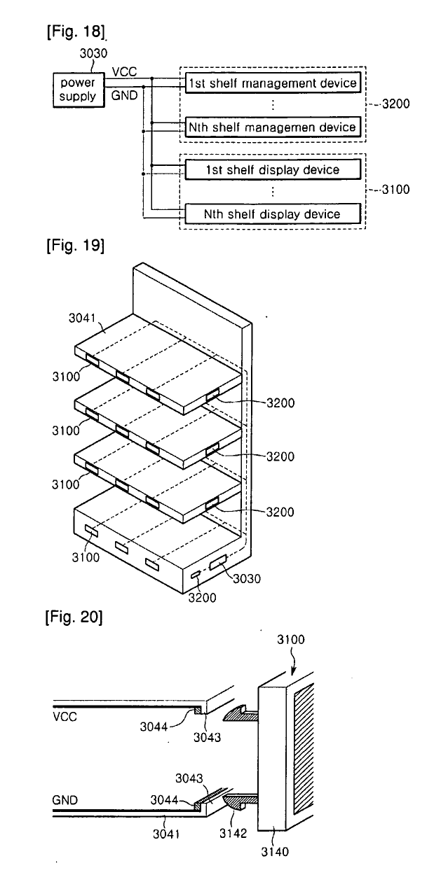

FIG. 18 is a circuit diagram showing a power supply of a shelf connected to the shelf management device and the shelf display device 3100 in the management system 3200 of the public facilities according to a third embodiment of the present invention. FIG. 19 is a view showing a shelf 3041 in which the shelf management device 3200 and the shelf display device 3100 are installed according to an embodiment.

The first image display device 3110 of the shelf display device 3100 displays multimedia data such as advertisement information and information about a bulletin board as well as simple character data. The first image display device 3110 may be realized by using a color LCD.

If the first image display device 3110 is realized by using the color LCD, a great amount of power is consumed. In addition, since many shelf display devices 3100 are mounted on one shelf 3041 as shown in FIG. 19, a battery cannot sufficiently supply power to the first image display device 3110.

Therefore, according to the embodiment, as shown in FIG. 18, an additional power supply 3030 is provided on the shelf 3041 of the store managing system.

One power supply 3030 can be installed in a shelf structure, and a power line Vcc and a grounding line GND are connected to the shelf management device 3200 and the shelf display device 3100 in parallel.

FIG. 19 is a view showing the shelf 3041 in the shelf management device 3200 and the shelf display device 3100 are installed in the management system of the public facilities according to the third embodiment of the present invention.

As shown in FIG. 19, the power supply 3030 is installed at a lower end of the shelf 3041 such that the power supply 3030 is easily connected to a power line of the store, and one shelf management device 3200 is installed one the shelf 3041.

In addition, a plurality of shelf display devices 3100 are installed on a side surface of the shelf 3041 provided at a side of a corridor. The power line Vcc and the grounding line GND connect the shelf management device 3200 and the shelf display device 3100 to the power supply 3030 through an internal corridor of the shelf 3041.

FIG. 20 is a view showing the assembled structure of the shelf display device 3100 and the shelf 3041 in the management system of public facilities according to the third embodiment of the present invention.

Since a width of a typical shelf is too narrow to install the shelf display device 3100, the shelf display device 3100 having various sizes cannot be attached to the shelf 3041

In addition, when the shelf display device 3100 is bonded to the shelf 3041 by using an adhesive member, the shelf display device 3100 cannot be detachable from the shelf 3041. Accordingly, the position of the shelf display devices 3100 cannot be adjusted according to the arrangement shape of the shelf display devices 3100.

Referring to FIG. 20, first coupling units 3142 are formed on a rear surface of a housing 3140 of the shelf display device 3100, and second coupling units 3043 are formed on a side surface of the shelf 3041 facing the corridor such that the first coupling units 3142 are detachable from the shelf display device 3100.

The first coupling unit 3142 has a protrusion shape, and is provided at an end thereof with a protrusion. The first coupling unit 3142 comprises metal.

Two first coupling units 3142 are provided. When the two first coupling units 3142 are coupled to the second coupling units 3043, the two first coupling units 3142 are connected to the power line Vcc and the grounding line GND of the power supply 3030 so as to be electrically connected to the shelf display device 3100, the image control device 3120, and the first ZigBee device 3130 provided in the housing 3140.

The second coupling units 3043 are provided on a side surface of the shelf 3041 facing the corridor and are prepared in the form of locking protrusions locked with the first coupling units 3142 at an upper and lower ends of an opening. Accordingly, the first coupling unit 3142 is introduced into the shelf 3041 through the opening, and the protrusion of the first coupling unit 3142 is locked with the second coupling unit 3043 such that the protrusion of the first coupling unit 3142 can be elastically supported by the second coupling unit 3043.

In addition, the second coupling units 3043 are provided at inner portions thereof with electrodes 3044 connected to the power line Vcc and the grounding line GND line, so that the first coupling unit 3142 is electrically coupled with the second coupling unit 3043. In other words, the first and second coupling unit 3142 and 3043 can be mechanically coupled with each other, and serve as electrical connectors.

Accordingly, a store manager can attach/detach the shelf display devices 3100 having various sizes to/from the shelf 3041, and adjust the position of the shelf display devices 3100.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

### INDUSTRIAL APPLICABILITY

The present invention is applicable for resource management of public facilities.

1. A management system of public facilities, the management system comprising:
a tag unit which transmits identification information of a target resource to be managed through a first network;
an intermediate managing unit which has intrinsic position information thereof, processes the identification information received through the first network, and transmits the identification information through a second network; and
a host unit which creates state information comprising a movement state of the target resource based on the intrinsic position information of the intermediate managing unit and the identification information of the target resource received through the second network.

2. The management system of claim 1, wherein the identification information of the tag unit is assigned to a vehicle, and the tag unit comprises a tag device transmitting the identification information through the first network;
the intermediate managing unit comprises a reader device, which generates tag analysis information by receiving the identification information of the vehicle through the first network, and a ZigBee device, which transmits/receives the identification information through the second network; and
the host unit generates movement information and parking state information of the vehicle by receiving the identification information from the intermediate managing unit.

3. The management system of claim 2, wherein the tag analysis information generated by the reader device of the intermediate managing unit comprises personal information of a driver and vehicle information.

4. The management system of claim 2, wherein the tag unit comprises a tag-ZigBee device which transmits/receives the identification information to/from at least one of the host unit and the intermediate managing unit through the second network, and
the host unit identifies the vehicle by matching the identification information transmitted from the tag unit with the tag analysis information transmitted from the intermediate managing unit and generates the movement information and the parking state information of the vehicle.

5. The management system of claim 4, wherein the intermediate managing unit generates parking state information related to the parking regions by performing a comparison operation with respect to a number of all parking regions in which the vehicle is parked, a number of tag units communicating with the intermediate managing unit through the tag-ZigBee device, and a number of tag units entered into a sleep mode after communication for a predetermined time,

6. The management system of claim 5, wherein the intermediate managing unit comprises an indicator indicating the parking state information of the parking regions.

7. The management system of claim 2, wherein the host unit creates a map for parking facilities by calculating coordinates for parking regions in which the vehicle is parked, obtains coordinates of an installation position of the intermediate managing unit, marks the coordinates on the map, and displays the movement information and the parking state information of the vehicle on a predetermined display unit by generating the movement information and the parking state information as the identification information of the vehicle is sequentially transferred to the host unit from the intermediate managing unit.

8. The management system of claim 2, wherein the host unit displays the movement information, the parking state information, and parking charge information of the vehicle on a predetermined display unit by generating the movement information, the parking state information, and the parking charge information of the vehicle.

9. The management system of claim 2, comprising a first sensor connected to the intermediate managing unit to detect quantity of light; and
an illuminator connected to the intermediate managing unit, wherein the intermediate managing unit controls the illuminator based on the movement information of the vehicle and detection information of the first sensor.

10. The management system of claim 1, wherein the identification information of the tag unit is provided to a patient or a staff member in a hospital, the tag unit comprises a tag device, which transmits the identification information through the first network, and a tag-ZigBee device which transmits/receives the identification information to/from at least one of the host unit and the intermediate managing unit through the second network;
the intermediate managing unit comprises a first reader device, which generates tag analysis information by receiving the identification information provided to the patient or the staff member through the first network, and an intermediate-ZigBee device, which transmits/receives the identification information through the second network; and
the host unit generates position information of at least one of the patient and the staff member by receiving the identification information.

11. The management system of claim 10, wherein the host unit determines a position of a movable bed having the first reader device based on the tag analysis information received through the second network.

12. The management system of claim 11, wherein the host unit generates patient transport information by using information about the patient and the position of the movable bed, generates patient handling information based on the patient transport information, and transmits at least one of the patient transport information, the patient handling information, and staff call information to the tag-ZigBee device; and
the tag unit outputs as multimedia data at least one of the patient transport information, the patient handling information, and the staff call information.

13. The management system of claim 10, comprising:
a second reader device which generates the tag analysis information by receiving identification information assigned to the patient through the first network; and
an information providing terminal which provides hospital operation information corresponding to the patient information to the patient by receiving the hospital operation information from the host unit.

14. The management system of claim 13, wherein the hospital operation information corresponding to the patient information comprises at least one of consultation guide information, facility guide information, parking guide information, settlement guide information, and prescription guide information.

15. The management system of claim 1, wherein the identification information of the tag unit is provided to at least one of an article, a cart, and a customer, and the tag unit comprises a tag device which transmits the identification information through the first network;
the intermediate managing unit comprises a reader device, which generates tag analysis information by receiving the identification information provided to at least one of the article, the cart, and the customer through the first network, and a ZigBee device which transmits/receives the identification information through the second network; and
the host unit generates at least one movement information of the article, article arrangement information, stored article information, and position information of the customer by receiving the identification information and using article routing information and article information.

16. The management system of claim 15, wherein the tag unit comprises a tag-ZigBee device transmitting/receiving the identification information to/from at least one the host unit and the intermediate managing unit through the second network; and
the host unit generates at least one of the article routing information and the position information of the customer by matching the identification information transmitted from the tag unit with the tag analysis information transmitted from the intermediate managing unit.

17. The management system of claim 15, wherein the intermediate managing unit comprises a shelf display device which displays at least one of advertisement information, sale information, discount information, information for sale based on order of arrival and the article arrangement information relative to the article by receiving the identification information from the tag unit through the ZigBee device.

18. The management system of claim 17, wherein a plurality of shelf display devices are mounted on a shelf according to a type of articles arranged on the shelf; and
a shelf management device is installed on the shelf in order to manage the shelf display devices by dividing the shelf display devices into several unit groups.

19. The management system of claim 15, wherein the reader device of the intermediate managing unit comprises a plurality of antennas installed in several regions of the shelf, the shelf is partitioned according to an installation position of the antenna having communicated with the tag device, if the antennas communicate with the tag device, the movement information of the article on the shelf is generated, and the ZigBee device transmits the movement information of the article on the shelf to the host unit; and
the host unit generates consumer propensity information by receiving the movement information of the article from the intermediate managing unit to calculate at least one of article recognition time and purchase determining time.

20. The management system of claim 15, wherein the intermediate managing unit comprises an illuminator controlled by the ZigBee device; and
a light sensor, which transmits a detected illuminance value in an installation area of the illuminator through the ZigBee device,
and wherein the host unit transmits illumination control information of the illuminator according to the detected illuminance value received through the ZigBee device.

21. The management system of claim 15, wherein the host unit determines that the article is stolen and traces information of the customer having the tag unit if a charge for the article is not paid within a predetermined time after the movement information of the article is created, or if the host unit matches the position information of the customer with the movement information of the article, so that the position information of the customer deviates from a map of a store, but the charge for the article is not paid.

22. A management system for public facilities, the management system comprising:
a tag unit which comprises a tag device transmitting identification information provided in at least one of an article, a cart, and a customer through a first network;
a host unit which generates at least one movement information of the article, article arrangement information, stored article information, and position information of the customer by receiving the identification information and using routing information of the article and article information;
an intermediate managing unit which comprises a reader device, which generates tag analysis information by receiving the identification information transmitted from the tag unit through the first network, and a ZigBee device which makes data communication with the host unit through a second network,
wherein the intermediate managing unit comprises:
a shelf comprising a first coupling unit comprising a power supply device supplying power and an electrode connected to the power supply device, the shelf on which articles are arranged; and
a shelf display device which receives the power from the electrode of the first coupling unit, and is coupled to the shelf by a second coupling unit detachable from a side coupling section of the shelf to display information received from the ZigBee device or the reader device.
